(19) United States
(12) Patent Application Publication
(10) Pub. No.: US 2024/0050235 A1
(43) Pub. Date: Feb. 15, 2024

(54) ANATOMICALLY SHAPED STEMLESS SHOULDER FOR TOTAL SHOULDER REPLACEMENT AND REVERSE TOTAL SHOULDER

(71) Applicant: Zimmer, Inc., Warsaw, IN (US)

(72) Inventors: Yang Wook Son, Granger, IN (US); Christine Mirja Mueri, Winterthur (CH); Lori Bush, Churubusco, IN (US)

(21) Appl. No.: 18/227,664

(22) Filed: Jul. 28, 2023

Related U.S. Application Data
(60) Provisional application No. 63/397,053, filed on Aug. 11, 2022.

(51) Int. Cl. A61F 2/40; A61F 2/30

(52) U.S. Cl. CPC A61F 2/4003; A61F 2/30; A61F 2/4014; A61F 2002/30329

(57) ABSTRACT

An apparatus including a humeral anchor configured to be mounted to a humerus and configured to receive a humeral component; wherein the humeral anchor includes a central hub having a plurality of fins extending therefrom, wherein the plurality of fins are arranged in an asymmetric pattern.

### CLAIM OF PRIORITY

This application claims the benefit of U.S. Provisional Patent Application Ser. No. 63/397,053, filed on Aug. 11, 2022, the benefit of priority of which is claimed hereby, and which is incorporated by reference herein in its entirety.

### TECHNICAL FIELD

The present application relates to an orthopedic system and specifically to a shoulder implant system.

### BACKGROUND

In a healthy shoulder, the proximal humerus is generally ball-shaped, and articulates within a socket formed by the scapula, called the glenoid, to form the shoulder joint. Some implant systems for the total replacement of the shoulder joint generally replicate the natural anatomy of the shoulder. Such implant systems can include a humeral component having a stem that fits within the humeral canal, and an articulating head that articulates within the socket of a glenoid component implanted within the glenoid of the scapula.

Reverse-type shoulder implant systems have been developed in which the conventional ball-and-socket configuration that replicates the natural anatomy of the shoulder is reversed, such that a concave recessed articulating component is provided at the proximal end of the humeral component that articulates against a convex portion of a glenosphere of a glenoid component.

The design of humeral implants for shoulder arthroplasty has evolved over the years. Whether standard or reverse, the humeral component of traditional shoulder arthroplasty is traditionally designed with a stemmed implant. However, complications with stemmed implants have been reported such as periprosthetic humeral fractures after shoulder arthroplasty and intra-operative difficulties during revision surgeries. Accordingly, stemless shoulder prosthesis have been introduced. The key feature of a stemless humeral anchor implant is a cementless metaphyseal fixation to reduce the potential risks associated with using a stemmed humeral implant. However, current stemless humeral anchor systems do not account for all patient population as the size and/or shape is not designed anatomically to account for asymmetry of proximal humeral bones where dense bone is located toward cortical bone. Moreover, currently available humeral anchor systems do not offer a primary reverse shoulder arthroplasty and/or convertibility from anatomic shoulder arthroplasty to reverse shoulder arthroplasty.

### OVERVIEW

To further illustrate apparatuses and methods disclosed herein, a non-limiting list of examples is provided here:

Example 1 is an apparatus including a humeral anchor configured to be mounted to a humerus and configured to receive a humeral component; wherein the humeral anchor includes a central hub having a plurality of fins extending therefrom, wherein the plurality of fins are arranged in an asymmetric pattern.

Example 2 is the apparatus of Example 1, wherein optionally the humeral component includes a humeral head configured to be mounted on the anchor.

Example 3 is the apparatus of Example 1, wherein optionally the humeral component includes a concave recessed articulating component including a humeral tray and bearing assembly.

Example 4 is the apparatus of any of Example 1-3, wherein optionally the anchor includes a porous metal.

Example 5 is the apparatus of any of Examples 1-4, wherein optionally the plurality of fins are sized and positioned to conserve humeral bone.

Example 6 is the apparatus of Example 1, wherein optionally the anchor is configured to selectively receive either a humeral head or a humeral tray.

Example 7 is the apparatus of any of Examples 1-6, wherein optionally the plurality of fins includes six fins.

Example 8 is the apparatus of Example 7, wherein optionally the six fins include a first fin which is a laterally positioned fin configured to be positioned proximate to a greater tubercle of the humerus, a second fin configured to be positioned proximate to a lesser tubercle, a fourth fin positioned 180 degrees from the first fin, a third fin which is centered between the second fin and the fourth fin, a fifth fin positioned such that there is an equal angle between the third fin and the fourth fin and the fourth fin and the fifth fin, and a sixth fin which is centered between the fifth fin and the first fin.

Example 9 is the apparatus of Example 8, wherein optionally the first fin and the second fin have an angle therebetween of about 75 degrees.

Example 10 is the apparatus of any of Example 7-9, wherein optionally two or more of the plurality of fins includes suture holes configured to receive a suture.

Example 11 is the apparatus of any of Examples 10, wherein optionally one or more of the plurality of fins includes a T-shaped cross section having a first portion extending from the central hub and a second portion perpendicular to the first portion.

Example 12 is the apparatus of any of Examples 1-11, wherein optionally one or more of the plurality of fins includes a straight shape including a single portion extending from the central hub.

Example 13 is the apparatus of any of Examples 1-12, wherein optionally each of the plurality of fins includes a tapered shape and a V-shaped bottom edge.

Example 14 is a system including a humeral anchor configured to be mounted to a humerus, the humeral anchor including a central hub defining a cylindrical hole; a humeral component configured to be mounted to the cylindrical hole of the humeral anchor; and a complementary component configured to be mounted to a glenoid cavity, wherein the humeral component is adapted to operate with the complementary component; wherein the humeral anchor includes a central hub having a plurality of fins extending therefrom, wherein the plurality of fins are arranged in an asymmetric pattern and have varying heights and radial lengths than other of the plurality of fins.

Example 15 is the apparatus of Example 14, wherein optionally the plurality of fins includes six fins, wherein a first fin includes a laterally positioned fin configured to be positioned proximate to a greater tubercle of the humerus, a second fin positioned about 70-90 degrees from the first fin and configured to be positioned proximate to a lesser tubercle, a fourth fin positioned 180 degrees from the first fin, a third fin which is centered between the second fin and the fourth fin, a fifth fin positioned such that there is an equal angle between the third fin and the fourth fin and the fourth fin and the fifth fin, and a sixth fin which is centered between the fifth fin and the first fin.

Example 16 is the apparatus of any of Examples 14-15, wherein optionally one or more of the plurality of fins includes a T-shaped cross section having a first portion extending from the central hub and a second portion perpendicular to the first portion.

Example 17 is the apparatus of any of Examples 14-16, wherein optionally each of the plurality of fins includes a tapered shape and a V-shaped bottom edge.

Example 18 is a method of implanting a humeral component including mounting a humeral anchor to a humerus, the humeral anchor including a central hub defining a cylindrical hole; and mounting a humeral component to the cylindrical hole of the humeral anchor; wherein the humeral anchor includes a plurality of fins extending from the central hub, wherein the plurality of fins are arranged in an asymmetric pattern and have varying heights and radial lengths than other ones of the plurality of fins.

Example 19 is the method of Example 18, wherein optionally the plurality of fins includes six fins, wherein a first fin includes a laterally positioned fin configured to be positioned proximate to a greater tubercle of the humerus, a second fin positioned about 70-90 degrees from the first fin and configured to be positioned proximate to a lesser tubercle, a fourth fin positioned 180 degrees from the first fin, a third fin which is centered between the second fin and the fourth fin, a fifth fin positioned such that there is an equal angle between the third fin and the fourth fin and the fourth fin and the fifth fin, and a sixth fin which is centered between the fifth fin and the first fin.

Example 20 is the method of any of Examples 18-19, further including mounting a complementary component to a glenoid cavity, wherein the humeral component is adapted to operate with the complementary component.

In Example 21, the apparatuses or methods of any one or any combination of Examples 1-20 can optionally be configured such that all elements or combinations of elements or options recited are available to use or select from.

These and other examples and features of the present apparatuses and methods will be set forth in part in the following Detailed Description. This Overview is intended to provide non-limiting examples of the present subject matter—it is not intended to provide an exclusive or exhaustive explanation. The Detailed Description below is included to provide further information about the present apparatus, systems, and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an arthroplasty system for a shoulder implant, in accordance with one embodiment.

FIG. 2 show an arthroplasty system for a reverse shoulder implant, in accordance with one embodiment.

FIG. 3 shows a top perspective view of a humeral anchor, in accordance with one embodiment.

FIG. 4 shows a bottom perspective view of the humeral anchor, in accordance with one embodiment.

FIG. 5 shows a top view of the humeral anchor, in accordance with one embodiment.

FIG. 6 shows a top view of the humeral anchor implanted into a resected humerus, in accordance with one embodiment.

FIG. 7 shows a cross-section of a central hub of the humeral anchor, in accordance with one embodiment.

FIG. 8 shows a cross-section of a fin of the humeral anchor, in accordance with one embodiment.

FIG. 9 shows a series of humeral anchors of varying sizes, in accordance with one embodiment.

FIG. 10 shows a perspective view of a humeral anchor being implanted into a resected surface of a humerus using a driver tool, in accordance with one embodiment.

FIG. 11 shows a method of mounting a humeral component, in accordance with one embodiment.

### DETAILED DESCRIPTION

As used herein, the following directional definitions apply. Anterior and posterior mean nearer the front or nearer the rear of the body, respectively, proximal, and distal mean nearer to or further from the root of a structure, respectively, and medial and lateral mean nearer the sagittal plane or further from the sagittal plane, respectively. The sagittal plane is an imaginary vertical plane through the middle of the body that divides the body into right and left halves.

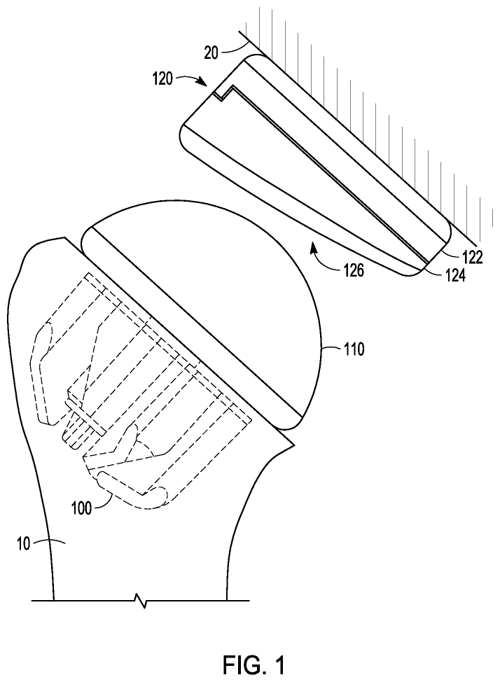

FIG. 1 shows an arthroplasty system for an anatomic shoulder implant, in accordance with one embodiment. In this example, the system includes a humeral anchor 100 configured to be mounted to a humerus 10. A humeral component, such as a humeral head 110 can be mounted to the anchor 100. As will be further discussed below, the humeral head 110 includes a post or cone, which can be a male taper, extending from a bottom surface which mounts to a cylindrical hole in the anchor 100. In another example, a humeral head adapter component can be used that includes two male tapers, one that interacts with the humeral anchor 100 and a second taper that couples to a mounting hole in the humeral head 110.

The system further includes a complimentary component, such as a concave recessed articulating component 120 including a tray 122 and a bearing 124. The bearing 124 includes a concave bearing surface 126 which articulates with the humeral head 110 after the implantation. The articulating component 120 is configured to be mounted to a scapula 20 proximate to a glenoid cavity. In other examples, the concave articulating component can include any concave bearing surface mounted proximate the glenoid cavity. For example, the concave articulating component can be mounted directly to the glenoid cavity without an intervening tray.

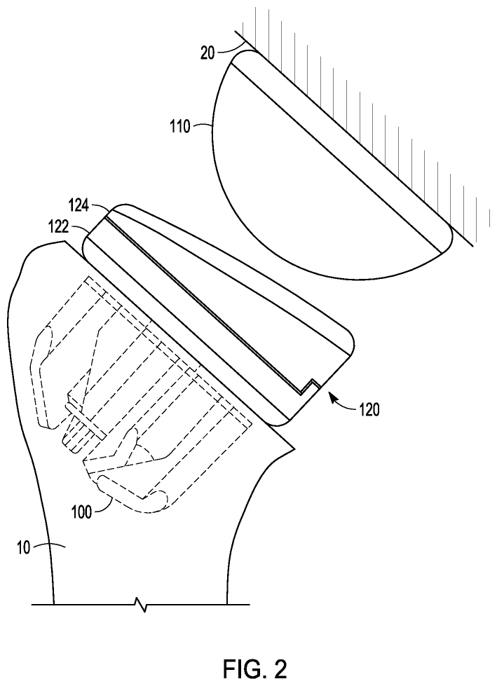

FIG. 2 shows an arthroplasty system for a reverse shoulder implant. Here the humeral head or a glenosphere 110 can be mounted to the scapula 20, while the articulating component 120 can be mounted to the humerus 10. Again, a post extending from a bottom of the tray 122 can be mounted to a hole located in the anchor 100. The glenosphere 110 can also be placed onto another component that can be mounted into the glenoid cavity. Thus, the glenosphere does not need to be directly attached to the scapula.

As noted above, currently marketed stemless humeral anchor implants do not account for all patient population as their size and/or shape is not designed anatomically to account for asymmetry of proximal humeral bones where dense bone is located toward cortical bone. Moreover, the currently available stemless humeral anchor systems do not offer a primary reverse shoulder arthroplasty and/or convertibility from anatomic shoulder arthroplasty to reverse shoulder arthroplasty.

Accordingly, the present system provides the stemless humeral anchor 100 designed for anatomical shape and fixation stability. In one example, the design of the humeral anchor 100 can be determined using methods described U.S. App. 63/312,212, filed Feb. 21, 2022, and which is incorporated herein by reference in its entirety. Moreover, the present system also allows a single humeral anchor system to be used for either anatomic or reverse total shoulder arthroplasty. The anchor 100 can be used as a primary reverse shoulder arthroplasty or convertible intra-operatively or in a revision situation.

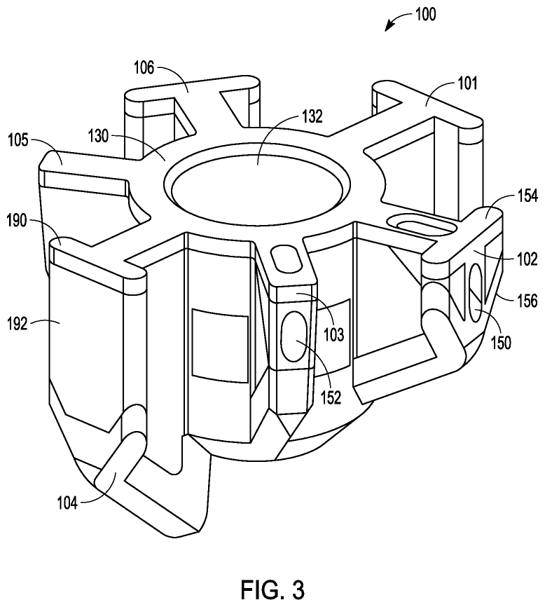

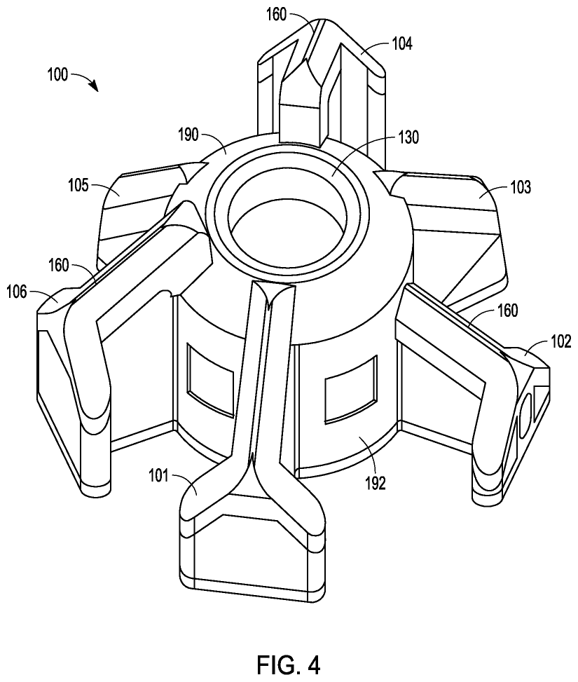

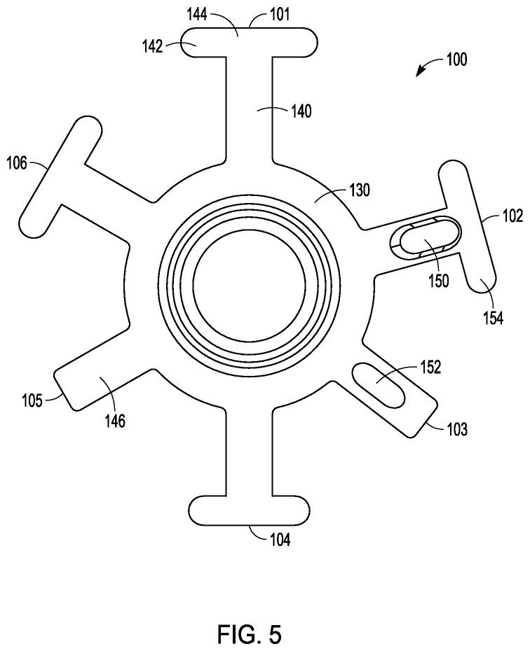

FIGS. 3, 4, and 5 show details of the humeral anchor 100, in accordance with one embodiment. FIG. 3 shows a top perspective view of the humeral anchor 100, FIG. 4 shows a bottom perspective view of the humeral anchor 100, and FIG. 5 shows a top view of the humeral anchor 100. The humeral anchor 100 of FIGS. 3-5 is for a left arm. For a right arm, the design of the anchor 100 would be a mirror image of the design shown.

In this example, the humeral anchor 100 includes a central hub 130 having a plurality of fins 101-106 extending radially from the outer surface walls of the central hub 130. The central hub 130 includes a cylindrical hole 132, which can include a female taper, configured to receive the post or cone of either the humeral head 110 or the articulating component 120. (FIGS. 1-2). In one example, the top surface of the humeral anchor 100 defines a planar surface where the central hub 130 and the plurality of fins 101-106 have co-planar upper surfaces. In other examples, the upper surfaces can be parallel, yet offset.

Here, each of the fins 101-106 can have a different height from their top surfaces to their respective bottom edges. Also, each of the fins 101-106 can have a varying radial length from the outer surface of the central hub 130 to their outermost surface. Moreover, the plurality of fins 101-106 can be arranged in an asymmetric pattern as viewed from the top of the humeral anchor 100. The sizes and positions of the plurality of fins 101-106 are designed to take advantage of the anatomy of the humerus without impinging on the cortical bone. For example, the fins 101-106 can be sized and positioned so that the fins are as long as possible without touching or impinging the cortical bone.

In one example, one or more of the plurality of fins 101-106 can include a T-shaped cross section. For example, in this embodiment, fins 101, 102, 104, and 106 include a T-shape. The T-shaped fins can include a first portion 140 extending from the central hub 130 and a second portion 142 perpendicular to the first portion 140. An outer flat surface 144 of the second portion 142 provides additional stability within the bone.

Further, one or more of the plurality of fins 101-106 can include a straight shape without the T-portion. For example, fins 103 and 105 can include a straight shape including a single portion 146 extending from the central hub 130. The fins without the T-shape can be located so as not to impinge on the cortical bone of the humerus.

In one embodiment, one or more of the plurality of fins 101-106 can include suture holes 150, 152 configured to receive a suture. For example, the suture holes 150, 152 can include a bore extending from a top surface 154 of the fin to an outer side surface 156 of the fin. In one example, two of the fins 102, 103 can include the suture holes 150, 152. The fins 102, 103 are on the side of the anchor 100 that will be mounted on the anterior side of the humerus so as to attach the suture to soft tissue.

In this embodiment, one or more of the plurality of fins 101-106 can include a V-shaped bottom edge 160. This sharp edge 160 at the bottom of the fins 101-106 allow for self-punching during implantation. Also, the bottom edge 160 of each of the fins 101-106 has a sloped shape where the section of the bottom edge 160 closest to the central hub 130 is lower than the outer tip of the fin so that the bottom edge slopes upward from the central hub 130 to the outer tip of the fin. Also, as noted above, each of the plurality of fins 101-106 can have a different radial length extending from the central hub 130 and can have different heights extending from a top surface of the fin to the bottom edge 160 of the fin.

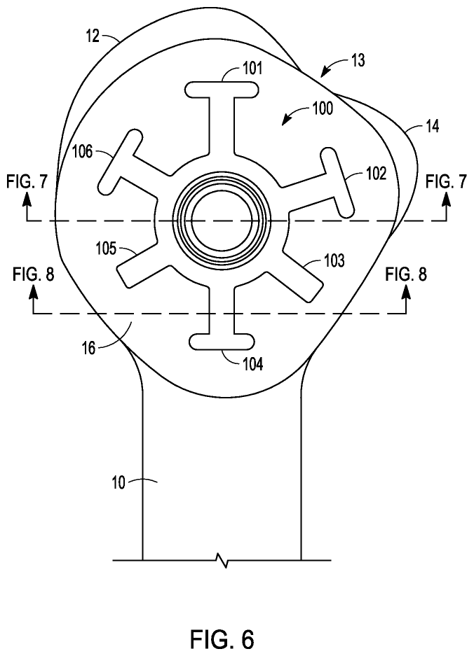

FIG. 6 shows a top view of the humeral anchor 100 implanted into a resected humerus 10, in accordance with one embodiment. In the example shown, the humerus 10 is a left arm humerus. The humeral anchor 100 is implanted so that the top surface of the humeral anchor 100 is flush with a surface of the resected humerus 10 at the initial insertion of the humeral anchor 100. When the mating component (humeral head or tray) is then assembled in-situ, the humeral anchor 100 will subside slightly deeper as there is a gap between the humeral anchor 100 and the mating component due to the taper connection. Here, the plurality of fins 101-106 of the humeral anchor 100 are sized and positioned to conserve humeral bone by avoiding cortical impingement while allowing the fins to be long enough to provide needed stability.

In this example, the humeral anchor 100 includes six fins 101-106. The six fins include a first fin 101 which is a laterally positioned fin positioned proximate to the greater tubercle 12 of the humerus 10, a second fin 102 is positioned so as to be proximate to a lesser tubercle 14. Alternatively, the first fin 101 and the second fin 102 can be equally spaced out from the bicipital groove 13 of the humerus. In this embodiment the angle between the first fin 101 and the second fin 102 is about 75 degrees. This angle covers the majority of humerus bones in the population regarding the angular spacing between the greater tubercle 12 and the lesser tubercle 14. In other examples, the angle between the first fin 101 and the second fin 102 can be about 60 degrees to about 100 degrees. In one embodiment, the angle can be in the range between about 70 to 90 degrees.

A fourth fin 104 can be positioned 180 degrees from the first fin 101, a third fin 103 can be centered between the second fin 102 and the fourth fin 104, a fifth fin 105 can be positioned such that there is an equal angle between the third fin 103 and the fourth fin 104 and the fourth fin 104 and the fifth fin 105, and a sixth fin 106 which can be centered between the fifth fin 105 and the first fin 101.

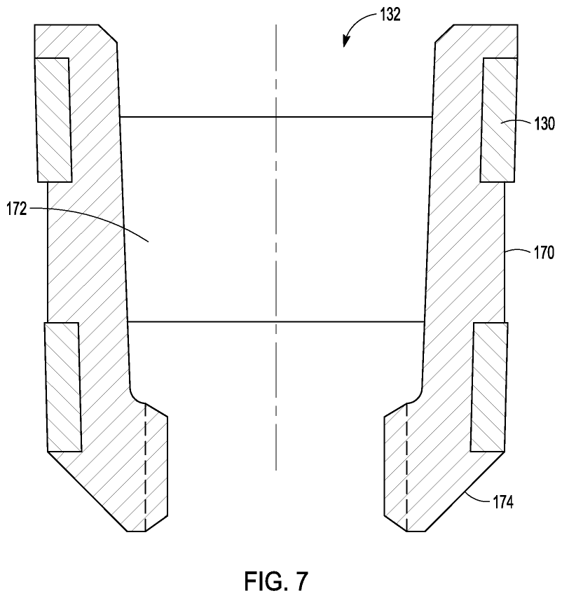

FIG. 7 shows a cross-section of the central hub 130, in accordance with one embodiment. The central hub 130 can include walls defining an outer surface 170 having a tapered shape. Likewise, inner walls 172 defining the central hole 132 also define a tapered shape configured to receive a humeral component. A bottom edge 174 of the central hub can be V-shaped to help during the implantation by providing a cutting edge.

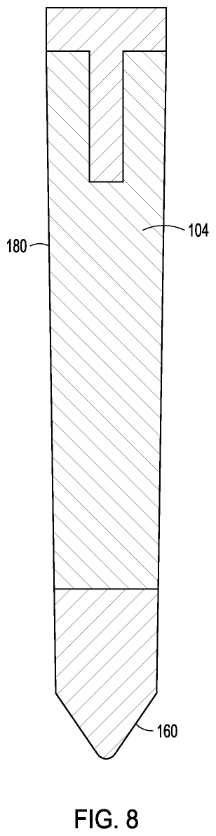

FIG. 8 shows a cross-section of the fin 104, in accordance with one embodiment. Each of the other of the plurality of fins 101-106 can include a similar structure. Here, the fin 104 includes an outer surface 180 having a tapered shape. As described above, the bottom edge 160 of the fin 104 can be V-shaped to help during implantation.

Referring again to FIGS. 3-4, the humeral anchor 100 can be formed of a titanium metal frame and can include porous metal portions or coatings for biologic bony ingrowth. For example, the anchor 100 can be manufactured utilizing additive manufacturing to incorporate a porous metal. A titanium frame 190 can be provided and a porous metal 192 can be added at certain portions, for example, at the outer surface of the central hub 130 and at the side walls and end surfaces of the fins 101-106. In this example, no porous metal is located on the bottom edges 160 of the fins or the bottom edge of the central hub 130 to allow for an easier revision process if needed.

Accordingly, in some examples, the humeral anchor 100 can be constructed of, or coated with, porous biomaterial to encourage bone growth into the implant. The need for cancellous bone substitute and/or cell and tissue receptive material is significant. Bone ingrowth into the voids of a porous material provides ideal skeletal fixation for permanent implants used for the replacement of bone segments damaged or lost due to any number of reasons, or for joint prostheses in some instances. Biological compatibility, intimate contact with the surrounding bone, and adequate stability during the early period of bone ingrowth have been identified as important requirements, along with proper porosity. One example of such material with biological compatibility and other traits is a porous titanium metal or metal alloy produced using OsseoTi™ porous metal technology generally available from Zimmer Biomet, Inc., of Warsaw, Ind.

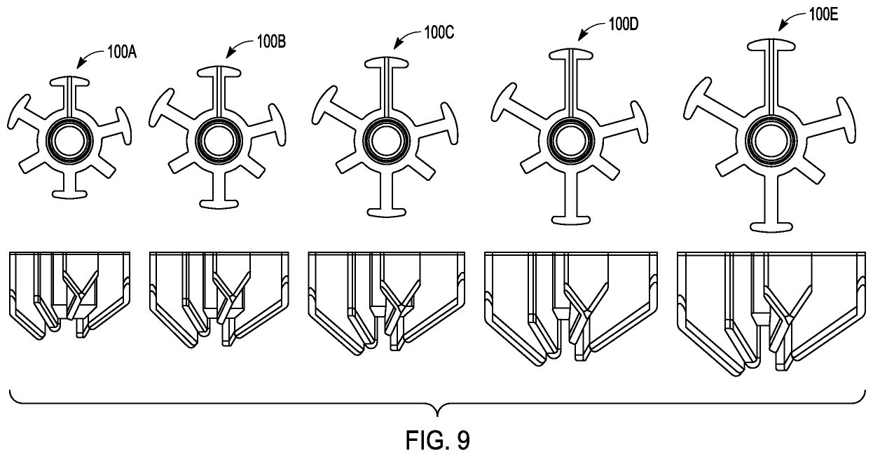

FIG. 9 shows a series of humeral anchors 100a-100e of varying sizes to be used for different size humans. Each of the anchors 100a-100e have similar proportions and overall structure as anchor 100 discussed above, only differing in their overall size.

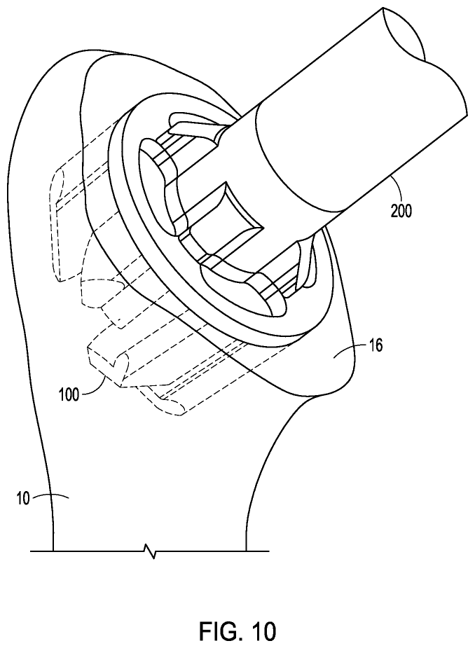

FIG. 10 shows a perspective view of a humeral anchor being implanted into a resected surface 16 of a humerus using a driver tool 200. As noted above, the sharp edge 160 at the bottom of the fins 101-106 (FIGS. 3-4) allow for self-punching during implantation.

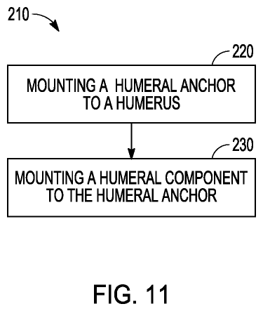

FIG. 11 shows a method (210) of implanting a humeral component to a humerus. For example, after the humerus has been properly resected, the driver tool 200 can be used for mounting a humeral anchor to a humerus (220), the humeral anchor including a central hub defining a cylindrical hole, such as a female taper. The method further includes mounting a humeral component (230) to the cylindrical hole of the humeral anchor. The humeral anchor can include a plurality of fins extending from the central hub, wherein the plurality of fins are arranged in an asymmetric pattern and have varying heights and radial lengths than other ones of the plurality of fins.

As discussed above, the plurality of fins can include six fins, wherein a first fin includes a laterally positioned fin positioned proximate to the greater tubercle of the humerus, a second fin positioned proximate to the lesser tubercle, a fourth fin positioned 180 degrees from the first fin, a third fin which is centered between the second fin and the fourth fin, a fifth fin positioned such that there is an equal angle between the third fin and the fourth fin and the fourth fin and the fifth fin, and a sixth fin which is centered between the fifth fin and the first fin.

The method (210) can further include mounting a complementary component to a glenoid cavity, wherein the humeral component is adapted to operate with the complementary component. The humeral component and the complementary component can either be a humeral head and a bearing or vice versa for a standard or a reverse shoulder arthroplasty. Accordingly, the present anchor system allows for either type, even during a revision or conversion.

The present humeral anchor design allows for a stemless shoulder implant where the anchor provides bone conservation while still providing stability. Accordingly, the present system helps account for a wide patient population as the size and/or shape is designed anatomically to account for asymmetry of proximal humeral bones where dense bone is located toward cortical bone. Moreover, the present system offers a primary reverse shoulder arthroplasty and/or convertibility from anatomic shoulder arthroplasty to reverse shoulder arthroplasty.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects. The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus comprising:
a humeral anchor configured to be mounted to a humerus and configured to receive a humeral component;
wherein the humeral anchor includes a central hub having a plurality of fins extending therefrom, wherein the plurality of fins are arranged in an asymmetric pattern.

2. The apparatus of claim 1, wherein the humeral component includes a humeral head configured to be mounted on the anchor.

3. The apparatus of claim 1, wherein the humeral component includes a concave recessed articulating component including a humeral tray and bearing assembly.

4. The apparatus of claim 1, wherein the anchor includes a porous metal.

5. The apparatus of claim 1, wherein the plurality of fins are sized and positioned to conserve humeral bone.

6. The apparatus of claim 1, wherein the anchor is configured to selectively receive either a humeral head or a humeral tray.

7. The apparatus of any of claim 1, wherein the plurality of fins includes six fins.

8. The apparatus of claim 7, wherein the six fins include a first fin which is a laterally positioned fin configured to be positioned proximate to a greater tubercle of the humerus, a second fin configured to be positioned proximate to a lesser tubercle, a fourth fin positioned 180 degrees from the first fin, a third fin which is centered between the second fin and the fourth fin, a fifth fin positioned such that there is an equal angle between the third fin and the fourth fin and the fourth fin and the fifth fin, and a sixth fin which is centered between the fifth fin and the first fin.

9. The apparatus of claim 8, wherein the first fin and the second fin have an angle therebetween of about 75 degrees.

10. The apparatus of claim 7, wherein two or more of the plurality of fins includes suture holes configured to receive a suture.

11. The apparatus of claim 1, wherein one or more of the plurality of fins includes a T-shaped cross section having a first portion extending from the central hub and a second portion perpendicular to the first portion.

12. The apparatus of claim 11, wherein one or more of the plurality of fins includes a straight shape including a single portion extending from the central hub.

13. The apparatus of claim 1, wherein each of the plurality of fins includes a tapered shape and a V-shaped bottom edge.

14. A system comprising:
a humeral anchor configured to be mounted to a humerus, the humeral anchor including a central hub defining a cylindrical hole;
a humeral component configured to be mounted to the cylindrical hole of the humeral anchor; and
a complementary component configured to be mounted to a glenoid cavity, wherein the humeral component is adapted to operate with the complementary component;
wherein the humeral anchor includes a central hub having a plurality of fins extending therefrom, wherein the plurality of fins are arranged in an asymmetric pattern and have varying heights and radial lengths than other of the plurality of fins.

15. The system of claim 14, wherein the plurality of fins includes six fins, wherein a first fin includes a laterally positioned fin configured to be positioned proximate to a greater tubercle of the humerus, a second fin positioned about 70-90 degrees from the first fin and configured to be positioned proximate to a lesser tubercle, a fourth fin positioned 180 degrees from the first fin, a third fin which is centered between the second fin and the fourth fin, a fifth fin positioned such that there is an equal angle between the third fin and the fourth fin and the fourth fin and the fifth fin, and a sixth fin which is centered between the fifth fin and the first fin.

16. The system of claim 15, wherein one or more of the plurality of fins includes a T-shaped cross section having a first portion extending from the central hub and a second portion perpendicular to the first portion.

17. The system of claim 16, wherein each of the plurality of fins includes a tapered shape and a V-shaped bottom edge.

18. A method of implanting a humeral component comprising:
mounting a humeral anchor to a humerus, the humeral anchor including a central hub defining a cylindrical hole; and
mounting a humeral component to the cylindrical hole of the humeral anchor;
wherein the humeral anchor includes a plurality of fins extending from the central hub, wherein the plurality of fins are arranged in an asymmetric pattern and have varying heights and radial lengths than other ones of the plurality of fins.

19. The method of claim 18, wherein the plurality of fins includes six fins, wherein a first fin includes a laterally positioned fin configured to be positioned proximate a greater tubercle of the humerus, a second fin positioned about 70-90 degrees from the first fin and configured to be positioned proximate a lesser tubercle, a fourth fin positioned 180 degrees from the first fin, a third fin which is centered between the second fin and the fourth fin, a fifth fin positioned such that there is an equal angle between the third fin and the fourth fin and the fourth fin and the fifth fin, and a sixth fin which is centered between the fifth fin and the first fin.

20. The method of claim 18, further comprising mounting a complementary component to a glenoid cavity, wherein the humeral component is adapted to operate with the complementary component.
